(19) United States
(12) Patent Application Publication
(10) Pub. No.: US 2026/0112463 A1
(43) Pub. Date: Apr. 23, 2026

(54) SYSTEMS AND METHODS FOR SECURELY MANAGING CLINICAL DATA IN AN UNIFIED PLATFORM

(71) Applicant: CliniOps Inc., Fremont, CA (US)

(72) Inventors: Avik Kumar Pal, Fremont, CA (US); Yerramalli Subramaniam, Plesanton, CA (US)

(21) Appl. No.: 18/921,523

(22) Filed: Oct. 21, 2024

(51) Int. Cl. G16H 10/20

(52) U.S. Cl. CPC G16H 10/20

(57) ABSTRACT

The present disclosure provides a unified clinical trial data management system comprising a centralized database configured to store data related to multiple aspects of clinical trials and a plurality of interconnected modules corresponding to specific clinical trial functions. The system includes a data linking mechanism that creates and maintains associations between data entries across different modules based on predefined rules and enforces data dependencies between modules to ensure data integrity and consistency. The system automatically creates corresponding entries or placeholders in linked modules upon data entry in a primary module, prevents certain actions in one module until prerequisite conditions are met in another module, and enables real-time data reconciliation between linked modules without manual intervention or data transfer between separate applications.

### TECHNICAL FIELD

The present subject matter described herein, in general, relates to the field of clinical trial management, and more particularly to establishment of a unified platform for streamlining and automating clinical trial processes across multiple functional modules.

### BACKGROUND

An eClinical system with disparate databases face significant technical challenges that can compromise data integrity, efficiency, and patient safety. When different modules within a the eClinical system operate on separate databases, data silos are created, leading to fragmentation and inconsistency across the system. This problem is exacerbated when data is manually entered into siloed modules, introducing the risk of human error, data duplication, and discrepancies between modules. For instance, if patient demographics are entered manually into both an input module and a separate adverse event tracking module, any discrepancy in data entry could lead to misidentification or incorrect association of adverse events with patients. In yet another example, when data is entered into the Electronic Data Capture module, and same stratification variables are entered into the Interactive Response Technology module to randomize study participants. Any discrepancies in the data entered between the Electronic Data Capture system module and Interactive Response Technology module could lead to incorrect randomization or improper group assignments, potentially affecting the study's balance and validity. This lack of data synchronization can result in delayed or inaccurate reporting, potentially masking safety signals that are critical for patient well-being. Moreover, when data is compartmentalized, it becomes challenging to conduct comprehensive analyses or generate holistic reports, as information must be manually aggregated from multiple sources, increasing the likelihood of oversight or misinterpretation. These technical limitations not only hamper the efficiency of clinical trial operations but also pose significant risks to patient safety by potentially obscuring important patterns or trends that could indicate safety concerns.

Current clinical trial management systems often suffer from a lack of interoperability and real-time data synchronization. Many existing solutions rely on periodic batch updates or manual data transfers between modules, leading to latency in data availability and increased risk of data inconsistencies. This delay in data propagation can result in decision-making based on outdated or incomplete information, potentially impacting trial outcomes and patient care. Additionally, the absence of a unified data model across modules makes it difficult to implement robust data validation and quality control measures, as each siloed database may have its own data structure and validation rules. This heterogeneity complicates the process of maintaining data integrity and traceability throughout the clinical trial lifecycle. Furthermore, current systems frequently lack sophisticated audit trial capabilities that can track changes across all modules in real-time, making it challenging to comply with regulatory requirements and conduct thorough investigations in case of data discrepancies or safety concerns. The limited integration between modules also hinders the implementation of advanced analytics and machine learning algorithms that could potentially identify complex patterns or predictive indicators relevant to patient safety and trial efficacy.

### SUMMARY

Before the present systems and methods for managing data in a clinical trial of patients are described, it is to be understood that this application is not limited to the particular systems, and methodologies described, as there can be multiple possible embodiments which are not expressly illustrated in the present disclosures. It is also to be understood that the terminology used in the description is for the purpose of describing the particular implementations or versions or embodiments only and is not intended to limit the scope of the present application.

This summary is provided to introduce aspects related to a system and a method for securely managing clinical data associated with a patient enrolled in a clinical trial. This summary is not intended to identify essential features of the claimed subject matter nor is it intended for use in determining or limiting the scope of the claimed subject matter.

In one implementation, method for securely managing clinical data associated with a patient, corresponding to a patient identifier is disclosed. The method comprises receiving the clinical data related to the patient identifier corresponding to the patient via at least one module out of a plurality of modules of a processing unit. Each of the plurality of modules is associated with a corresponding data table within a single database communicably coupled to the processing unit. The clinical data received by the at least one module is stored in a standardized format against the patient identifier in the corresponding data table of the single database. Further, at least one relational link is dynamically created between the at least one module and one or more modules of the plurality of modules of the processing unit based on a set of preconfigured validation rules. Creating the relational link comprises associating the corresponding data tables of the at least one module and the one or more modules using the unique identifier. Further, information across the linked one or more modules is synchronized, in real time, based on the received clinical data from the at least one module. Such that synchronizing comprises automatically populating the data tables of the linked one or more modules based on the clinical data received by the at least one module and the set of preconfigured validation rules. The one or more metrics is generated corresponding to the patient identifier based on the synchronized information, from the data tables of the single database, corresponding to linked one or more modules and finally at least one of the generated one or more metrics corresponding to the patient identifier is displayed, on a user interface communicably coupled to the processing unit.

In one implementation, a system for securely managing clinical data associated with a patient, corresponding to a patient identifier is disclosed. The system comprises a memory and a processor communicatively coupled to the memory. The processor upon execution of the or more instructions stored in the memory is configured to receive the clinical data related to the patient identifier corresponding to the patient via at least one module out of a plurality of modules of a processing unit. Each of the plurality of modules is associated with a corresponding data table within a single database communicably coupled to the processing unit. The clinical data received by the at least one module is stored in a standardized format against the patient identifier in the corresponding data table of the single database. Further, at least one relational link is dynamically created between the at least one module and one or more modules of the plurality of modules of the processing unit based on a set of preconfigured validation rules. Creating the relational link comprises associating the corresponding data tables of the at least one module and the one or more modules using the unique identifier. Further, information across the linked one or more modules is synchronized, in real time, based on the received clinical data from the at least one module. Such that synchronizing comprises automatically populating the data tables of the linked one or more modules based on the clinical data received by the at least one module and the set of preconfigured validation rules. The one or more metrics is generated corresponding to the patient identifier based on the synchronized information, from the data tables of the single database, corresponding to linked one or more modules and finally at least one of the generated one or more metrics corresponding to the patient identifier is displayed, on a user interface communicably coupled to the processing unit.

### BRIEF DESCRIPTION OF THE DRAWINGS

The foregoing detailed description of embodiments is better understood when read in conjunction with the appended drawings. For the purpose of illustrating of the present subject matter, an example of a construction of the present subject matter is provided as figures, however, the invention is not limited to the specific method and system for securely managing clinical data through one or more modules of the system and generating real-time one or more metrics corresponding to a patient identifier during a clinical trial is disclosed in the document and the figures.

The present subject matter is described in detail with reference to the accompanying figures. In the figures, the left-most digit(s) of a reference number identifies the figure in which the reference number first appears. The same numbers are used throughout the drawings to refer to various features of the present subject matter.

FIG. 1 illustrates a network implementation for securely managing clinical data associated with a patient enrolled in a clinical trial, in accordance with an embodiment of the present disclosure.

FIG. 2 illustrates one or more modules of the system, in accordance with an embodiment of the present disclosure.

FIG. 3 (3A and 3B) illustrates a block diagram for a method for securely managing clinical data associated with a patient enrolled in a clinical trial, in accordance with an embodiment of the present disclosure.

FIG. 4 illustrating a protocol adherence mechanism, in accordance with an embodiment of the present disclosure.

The figures depict an embodiment of the present disclosure for purposes of illustration only. One skilled in the art will readily recognize from the following discussion that alternative embodiments of the structures and methods illustrated herein may be employed without departing from the principles of the disclosure described herein.

### DETAILED DESCRIPTION

Some embodiments of this disclosure, illustrating all its features, will now be discussed in detail. The words “receiving,” “storing”, “creating”, “synchronizing”, “generating”, “displaying”, and other forms thereof, are intended to be open ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise. Although any system and methods similar or equivalent to those described herein can be used in the practice or testing of embodiments of the present disclosure, the exemplary, system and methods are now described.

The disclosed embodiments are merely examples of the disclosure, which may be embodied in various forms. Various modifications to the embodiment will be readily apparent to those skilled in the art and the generic principles herein may be applied to other embodiments. However, one of ordinary skill in the art will readily recognize that the present disclosure is not intended to be limited to the embodiments described but is to be accorded the widest scope consistent with the principles and features described herein.

The proposed system addresses the above discussed technical challenges by implementing a centralized or a single database architecture with interlinked modules, eliminating data silos and manual data transfer processes. In this innovative approach, all modules of the clinical trial management system are connected to a single, unified database, ensuring data consistency and real-time synchronization across the entire system. When data is entered or updated in any module, the system automatically triggers relevant processes in interconnected modules, activating workflow sequences without manual intervention. For example, the entry of a new adverse event in an input module would immediately update the patient's record in another relevant module and trigger appropriate notifications in for example regulatory compliance module. This seamless data flow not only reduces the risk of data entry errors but also ensures that all stakeholders have access to the most up-to-date information at any given time. The system employs advanced preconfigured validation rules and cross-module integrity checks to maintain data quality and consistency. Furthermore, a comprehensive system tracks all data modifications across modules, providing a clear history of changes and supporting regulatory compliance efforts. By centralizing data and interlinking modules, the system enables sophisticated real-time analytics and reporting capabilities, facilitating early detection of safety signals and trend analysis. This integrated approach significantly enhances the efficiency of clinical trial management, improves data quality, and ultimately contributes to better patient safety outcomes by providing a holistic, real-time view of all trial-related data.

The present subject matter discloses a method and a system for securely managing clinical data associated with a patient enrolled in a clinical trial. In operation, the proposed system and method is designed to receive and store clinical data related to a patient identifier via at least one module out of a plurality of modules of a processing unit. Each of these modules is associated with a corresponding data table within the centralised/single database. In an embodiment, the patient identifier is pseudorandomized. The pseudorandomized patient identifier may be generated using a pseudo randomization algorithm that assigns a unique, random code to each patient. This identifier ensures that the patient's actual identity is obscured while still allowing the system to maintain a link to their clinical data. In an embodiment, the pseudorandomized patient identifier may be securely stored in a database alongside the associated clinical data, ensuring that all information is encrypted and protected against unauthorized access. The system employs strict access controls and encryption protocols to safeguard the data.

The system is further configured to dynamically create relational links between the modules based on a set of preconfigured validation rules. These links associate the corresponding data tables of the modules using the unique identifier, enabling real-time synchronization of information across the linked modules based on the received clinical data. The system also includes a feature of generating metrics corresponding to the patient identifier based on synchronized information from the data tables of the linked modules. These metrics can be displayed on a user interface communicably coupled to the processing unit, providing a comprehensive view of the patient's clinical data. This approach to manage the clinical trial data offers potential advantages in terms of efficiency, data integrity, and ease of use. By integrating various aspects of clinical trial management into a single platform, the system helps to streamline the process of conducting clinical trials, reduce the risk of data loss or inconsistencies, and facilitate more effective clinical outcome assessments and enhance patients'safety.

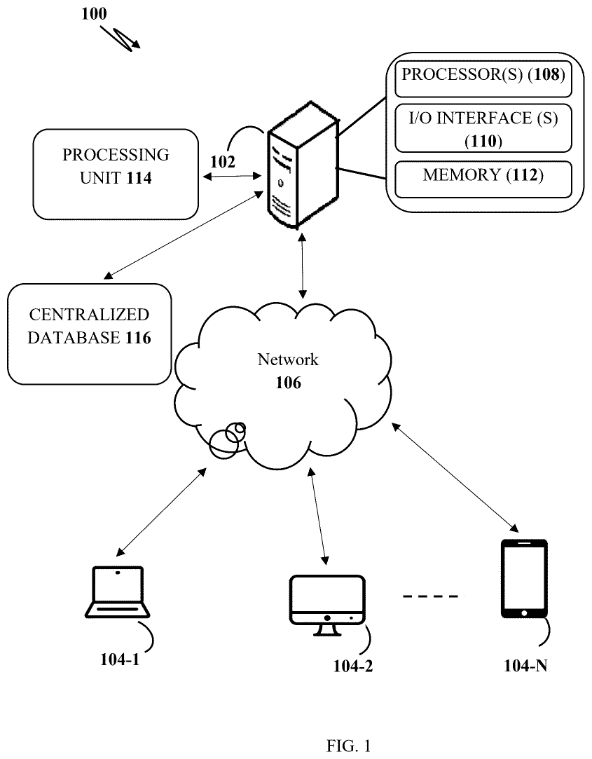

Referring now to FIG. 1, a network 100 implementation for securely managing clinical data through one or more modules of a processing unit during a clinical trial. The network 100 includes a system 102, one or more user devices 104-N (for example but not limited to one or more user devices 104-1, 104-2. 104-N) associated with one or more users, a processing unit 114, and a centralised database 116. Although the present disclosure is explained considering that the system 102 is implemented on a server, it may be understood that the system 102 may be implemented in a variety of computing systems, such as a laptop computer, a desktop computer, a notebook, a workstation, a virtual environment, a mainframe computer, a server, a network server, a cloud-based computing environment. In one implementation, the system 102 may be implemented in a cloud-based computing environment in which the system is configured to execute or interact with remotely located applications. Further, system 102 and user device 104 may communicate through the network 106. Examples of the user devices 104 may include, but are not limited to, a portable computer, a personal digital assistant, a handheld device, and a workstation.

In one implementation, the network 106 may be a wireless network, a wired network, or a combination thereof. The network 106 can be implemented as one of the different types of networks, such as intranet, local area network (LAN), wide area network (WAN), the internet, and the like. The network 106 may either be a dedicated network or a shared network. The shared network represents an association of the different types of networks that use a variety of protocols, for example, Hypertext Transfer Protocol Secure (HTTPS), Transmission Control Protocol/Internet Protocol (TCP/IP), Wireless Application Protocol (WAP), and the like, to communicate with one another. Further the network 106 may include a variety of network devices, including routers, bridges, servers, computing devices, storage devices, and the like.

In one embodiment, the system 102 may include at least one processor 108, an input/output (I/O) interface 110, a memory 112, and one or more modules explained later in the description. The at least one processor 108 may be implemented as one or more microprocessors, microcomputers, microcontrollers, digital signal processors, Central Processing Units (CPUs), state machines, logic circuitries, and/or any devices that manipulate signals based on operational instructions. Among other capabilities, the at least one processor 108 is configured to fetch and execute computer-readable instructions stored in the memory 112.

The I/O interface 110 may include a variety of software and hardware interfaces, for example, a web interface, a graphical user interface, wearables and the like. The I/O interface 110 may allow the system 102 to interact with the user directly or through the client devices 104. Further, the I/O interface 110 may enable the system 102 to communicate with other computing devices, such as web servers and external data servers (not shown). The I/O interface 110 can facilitate multiple communications within a wide variety of networks and protocol types, including wired networks, for example, LAN, cable, bluetooth etc., and wireless networks, such as WLAN, cellular, or satellite. The I/O interface 110 may include one or more ports for connecting a number of devices to one another or to another server.

The memory 112 may include any computer-readable medium or computer program product known in the art including, for example, volatile memory, such as static random-access memory (SRAM) and dynamic random-access memory (DRAM), and/or non-volatile memory, such as read only memory (ROM), erasable programmable ROM, flash memories, hard disks, Solid State Disks (SSD), optical disks, and magnetic tapes. The memory 112 may include routines, programs, objects, components, data structures, etc., which perform particular tasks or implement particular abstract data types. The memory 112 may include programs or coded instructions that supplement applications and functions of the system 102. In one embodiment, the memory 112, amongst other things, serves as a repository for storing data processed, received, and generated by one or more of the programs or the coded instructions.

In an embodiment, the system 102 may be communicatively coupled with a processing unit 114. In an embodiment, the processing unit may reside within the system. In yet another embodiment, the processing unit may reside outside the system 102. In an embodiment the processing unit has one or more modules residing on it. Each module may handle distinct types of data and perform specialized functions related to the clinical trial. The data is processed by one or more modules and is stored in the centralized database 116. The centralised database corresponds to one single database of the system, where all the data from the one or more modules is stored. By centralizing data, the centralized database 116 ensures consistency and synchronization across all modules. This means that any updates, changes, or new data entries made in the one or more modules are immediately reflected across the centralized database. In an embodiment, the centralized database 116 stores one or more clinical site information, patient information, visit schedules, and workflow parameters. The clinical site information may include details regarding various clinical trial sites, including their locations, capacities, contact details, and operational status. The patient information may include medical histories, demographic details, consent forms, treatment logs, and the like. The visit schedules may include timetables and schedules for patient visits, follow-ups, and other trial-related appointments to ensure smooth coordination and adherence to the trial protocol. The workflow parameters may include workflow configurations and parameters that define the operational procedures, data collection methods, and analysis protocols for the clinical trial, facilitating standardized processes and efficient management. In an embodiment, the centralised database may be a part of the system. In yet another embodiment, the centralised database may be a separate independent entity.

In an embodiment, the centralized database 116 may be structured to optimize data storage and retrieval for multiple clinical trial modules. The database may be designed to accommodate a wide range of data types and formats, reflecting the diverse nature of data collected in clinical trials. This may include structured data such as patient demographics, unstructured data such as free-text patient reports, and semi-structured data such as lab results. The database may be organized into a series of data tables, each data table is associated with a specific clinical trial module. For example, there may be a data table for the EDC module that stores raw data collected from electronic case report forms, a data table for the ePRO module that stores patient-reported outcome data, and a data table for the IRT module that stores randomization and drug dispensation data. Each data table may be designed to store data in a format that is optimized for the specific type of data it handles, facilitating efficient data storage and retrieval.

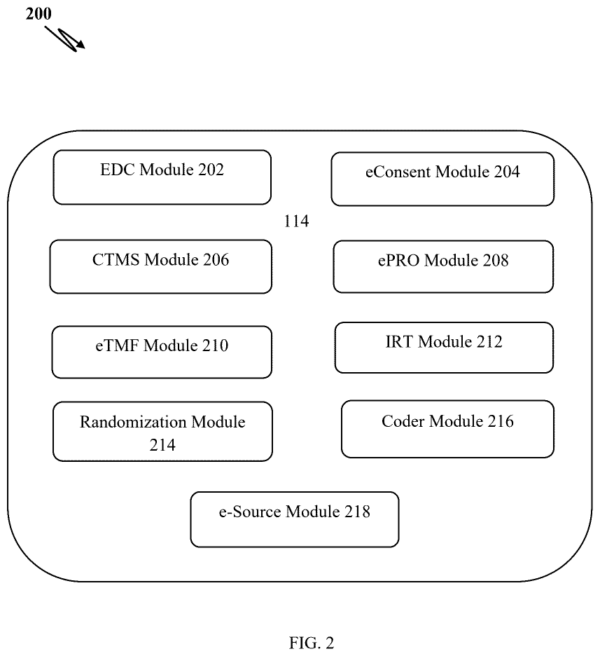

Referring to FIG. 2, in an embodiment, the system may include a plurality of integrated clinical trial modules, each designed to handle a specific aspect of clinical trial management. For example, the processing unit 114 may include an EDC (Electronic Data Capture) module 202. In an embodiment, the EDC module is configured to receive clinical data. The clinical data may include information associated with an event corresponding to a patient of the clinical trial. For example, event associated with the patient may be an adverse event, like abnormal blood pressure or temperature, or any other symptom that the patient may be facing. The patient may input the data associated with any symptom via the EDC module. In an embodiment, the clinical data may include data associated with any event occurring to the patient, demographic data, patient information, clinical assessments, laboratory results, and other relevant study data. The system secure stores all the data input to the EDC in the centralized database 116. This centralization allows for efficient data access, backup, and recovery. In an embodiment, the EDC module may receive non-clinical data. For example, such as patient-reported outcomes (PROs), lifestyle information, or data from wearable devices monitoring physical activity or sleep patterns. This non-clinical data may provide valuable insights into the patient's overall well-being and may complement the clinical data to offer a more comprehensive view of the patient's condition. In an embodiment, the system may also receive environmental data, such as location-based information or external factors like weather conditions, which could influence the patient's health. All non-clinical data is securely stored alongside clinical data in the centralized database 116, ensuring seamless integration and availability for analysis.

In an embodiment, the processing unit may include eConsent module 204. An eConsent module is used in the clinical trial to obtain informed consent from participants. This module may digitize the consent process, allowing participants to review, understand, and sign consent forms electronically. For example, the eConsent module may include multimedia elements such as videos, diagrams, and interactive questionnaires that help participants better understand the trials'purpose, procedures, risks, and benefits. The system may store the eConsent forms or consent data may be stored in the centralized database 116. The system may track the consent process in real-time, ensuring that all participants have completed the necessary consent forms before proceeding with the clinical trial. In an embodiment, the eConsent allows participants to complete the consent process remotely. The system may track the time stamp associated with activities performed by the patients on the eConsent form.

In an embodiment, the processing unit may include CTMS (Clinical Trial Management System module) 206 module. The CTMS module is designed to manage and streamline various aspects of the clinical trial. The CTMS Module encompasses a range of functionalities that support the planning, tracking, and management of the clinical trial, thereby integrating various components and processes into a cohesive system. In an embodiment, the processing unit may include an ePRO 208 module. An ePRO module stands for electronic Patient-Reported Outcome. The ePRO module is used to collect, manage, and analyse data directly reported by patients participating in the clinical trial. The ePRO module may display the patient's data on a dashboard. In an embodiment, the patient-reported data can be visualized using graphs and charts, allowing both patients and researchers to track symptoms, side effects, and overall health trends over time. The dashboard displays the patient's data in real time.

In an embodiment, the processing unit may include eTMF (electronic Trial Master File) module 210. The eTMF module is a critical component of the system, designed to manage and store essential documents related to the conduct of a clinical trial. This module serves as a digital repository for all trial-related documentation, ensuring regulatory compliance and facilitating efficient trial management. The eTMF module provides secure storage for all trial-related documentation in a structured, searchable format, with robust version control and audit trial capabilities. The eTMF module may support various file formats and aligns with industry standards and regulatory requirements. The eTMF module may integrate with other modules, allowing for efficient import and export of documents as needed for regulatory submissions or audits.

In an embodiment, the processing unit may include Interactive Response Technology (IRT) module 212 for managing patient randomization, treatment allocation, and drug supply logistics. The module provides real-time, interactive capabilities to support key trial operations. The IRT module implements various randomization schemes and can accommodate adaptive trial designs with dynamic algorithms. The module may automate treatment assignment based on randomization results and manages treatment kits or medication numbers, including blinding and unblinding procedures. The module tracks drug inventory across multiple sites, triggers automated resupply, and manages drug expiration dates and lot numbers. The module may generate patient visit schedules based on protocol specifications, including reminders and alerts for upcoming or overdue visits. The module may provide real-time status reports on patient enrolment, treatment allocation, and drug supply forecasting. It adheres to regulatory requirements and incorporates robust data security measures.

In an embodiment, the processing unit may include Coder Module 216. The Coder module plays a crucial role in standardizing and categorizing medical terminology. It serves as a bridge between the raw data entered in the Electronic Data Capture (EDC) system and standardized medical dictionaries. The coder module stores standardized terms, codes, and hierarchies from medical dictionaries like MedDRA and WHODrug, as well as the mappings between these standardized terms and the verbatim terms entered in the EDC. This standardization is essential for regulatory compliance, accurate reporting, and meaningful statistical analysis of clinical trial data. In an embodiment, each of the module may correspond to a separate data table in the centralised module, where the system stores data received by each of the module.

In an embodiment, the processing unit may include an e-source Module 218. In an embodiment, the eSource module is used for direct, real-time capture and management of clinical trial data from electronic sources. This module may interface with diverse data streams including electronic health records (EHRs), wearable devices, and patient-reported outcomes, employing standardized data formats (e.g., HL7 FHIR, CDISC ODM) to ensure interoperability. The e-source module may utilize advanced data validation algorithms and machine learning techniques to enhance data quality and detect anomalies in real-time. The eSource module may implement end-to-end encryption, role-based access controls, and multi-factor authentication to safeguard patient information and maintain compliance with regulations such as HIPAA and GDPR. This comprehensive approach significantly reduces data entry redundancies, accelerates study timelines, and enhances the overall efficiency and reliability of the clinical trial process.

In some embodiments, the processing unit 114 may include a notification system. The notification system may be designed to generate notifications based on data entered in the one or more modules. For instance, when a user completes a task in one of the modules, the notification system may generate a notification to inform other users or system of this completion. The user may correspond to any one or more of patient, doctor, nurse, researcher, admin staff, any personnel associated with the clinical trial. This notification system may help coordinate activities across the integrated modules, ensuring that all stakeholders are kept informed of trial progress. In an embodiment, the notification system may integrate with various communication channels to deliver notifications. For instance, the system may integrate with messaging apps, allowing it to send notifications directly to users'messaging inboxes. This could provide a convenient and immediate way for users to receive notifications, potentially improving response times and trial efficiency. In other cases, the system may integrate with SMS services, enabling it to send text message notifications to users'mobile phones. This could be particularly useful for users who do not have regular access to internet or email, ensuring that they can still receive important trial notifications.

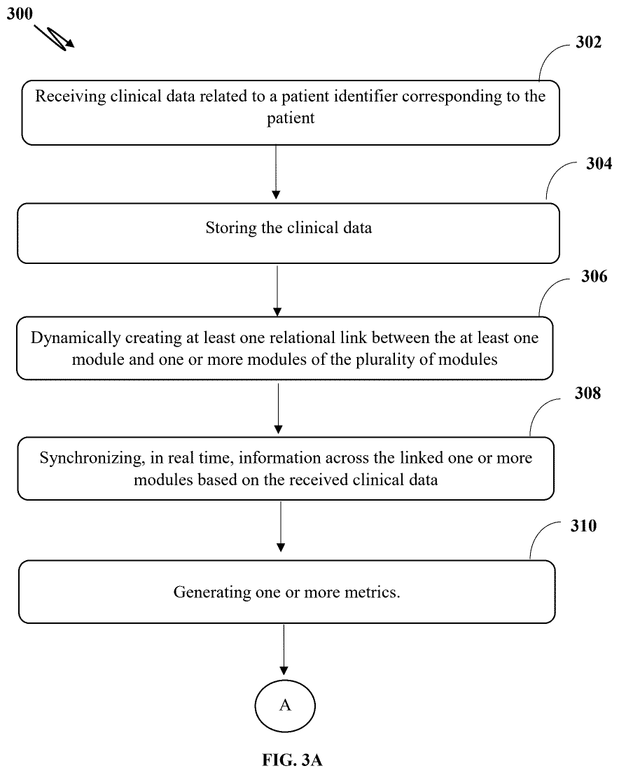

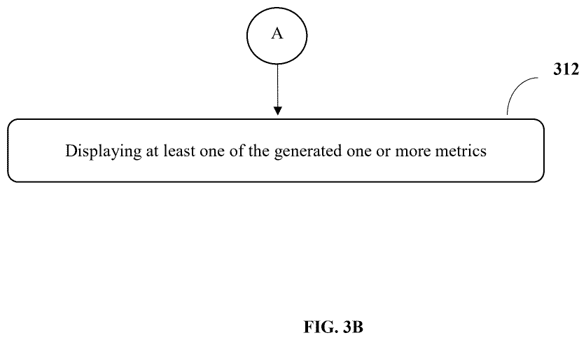

Referring now to FIG. 3 (3A and 3B), a method 300 for securely managing clinical data associated with a patient enrolled in a clinical trial, is shown, in accordance with one or more embodiments of the present subject matter. The method 300 may be described in the general context of computer executable instructions. Generally, computer executable instructions can include routines, programs, objects, components, data structures, procedures, modules, functions, etc., that perform particular functions or implement particular abstract data types.

The method 300 for securely managing clinical data during the clinical trial may be implemented in any suitable hardware, software, firmware, or combination thereof. However, for ease of explanation, in the embodiments described below, the method 300 may be considered to be implemented in the network 100 of FIG. 1 by the processor(s) 108 of the system 102 in association with the other devices in the network 100.

In an embodiment, referring to FIGS. 3A and 3B, at step 302, the system may be configured to receive clinical data associated with a patient identifier via a module of one or more modules. Such that each of the module is associated with a corresponding data table within the centralised database. In an embodiment, the clinical data corresponds to information related to an event associated with the respective patient. The event may be for example like raising or lowering blood pressure, raising or lowering temperature, or fluctuations in any vitals a patient corresponding to the patient identifier. In an embodiment, the event may be an adverse event or a non-adverse event. The clinical data may be received via one of the modes like online form, google form, web-form, mobile application, wearable devices, interactive voice systems, or in-person interview. In an embodiment, the patient may be given an option to select a mode to share the clinical data. In yet another embodiment, the system may not provide any option for selecting a mode and may provide a predetermined mode to enter the clinical data. For example, the EDC module may receive the clinical data. The EDC module may have a corresponding data table in which the system may store all the received clinical data. The data table may reside in the centralised database. Upon initiation of the data entry process via the EDC module, the system may identify the EDC module to be in an ‘active’ state. As clinical data is received, the system continuously updates the internal state of the EDC module to reflect the progress of data entry. Once all required clinical data fields have been populated and validated, the system changes the module's state to ‘completed.’

In an embodiment, the system may monitor if each of the populated field in the respective mode to receive the clinical data is completed. For example, the system may associate a time window with the respective module to enter the data. For example, the respective mode via which the clinical data may be received is enabled for a predetermined period of time. Like, if the mode of receiving the clinical data is an online form. The system may associate a time window with the respective online form and after the predetermined time the form will be deactivated, and the patient will not be able to enter the data in the form. In an embodiment, upon completion of the reception of the clinical data, the system may generate and send notification to relevant parties indicating that the clinical data have been entered. The relevant parties may be doctors, nurses, researchers, other users associated with the clinical trial. This feature ensures timely collection of data while maintaining compliance with legal and ethical standards.

In an embodiment, the system may allow authorized administrators to configure the time window based on factors such as legal requirements, institutional policies, the nature of the data, and patient-specific considerations. In yet another embodiment the system may predefine the time window based on predefined rules for each module. The time window may be defined by a start time/date, an end time/date, and a specified duration. Activation of the time window may occur when a module is enabled. During the active window, the system grants the patient access to the specific consent form, demographic form, or any other form/or other mode of reception of data and provides visual or audio cues indicating the remaining time for submission. In an embodiment, to account for potential time zone differences, all time windows are stored and processed in a standardized time format and displayed in the patient's local time zone. In an embodiment, the system may handle partial completions, saving any entered data but marking it as incomplete if the patient doesn't finish within the allotted time. In an embodiment, the system sends automated reminders to patients (or any other person filing the details via forms) at key points during the window to encourage timely completion. This time-bounded approach ensures that data is obtained in a timely manner, supports efficient healthcare operations, and maintains a clear record of the data, all while respecting patient autonomy and decision-making processes.

In an embodiment, the clinical data may be entered in at least one of the modules, via a user device interface using digital forms, e-signature capabilities, options for attaching scanned physical documents, and multi-language support for the forms. The system may continuously monitor and validate the entered data in real time. For example, the system may check if all required fields are completed, signatures or acknowledgments are properly recorded, date and time stamps are accurately logged, and any conditional fields (based on demographic data) are appropriately presented and captured.

In an embodiment, at step 304, the system may be configured to store the clinical data received by the at least one module in a standardized format against the patient identifier in the corresponding data table of the single database. This standardization process may involve several key components and considerations. The system may employ a consistent data structure across all modules, ensuring that data is stored in a uniform manner regardless of its source. This standardized format may include predefined fields, data types, and relationships, facilitating easier data integration, retrieval, and analysis. In some cases, the standardization process may involve data normalization. This may include breaking down complex data structures into simpler, more manageable components. For example, a patient's address may be stored as separate fields for street, city, state, and zip code, rather than as a single text string. The system may utilize a common set of controlled vocabularies or terminologies across all modules. This may include standardized medical terminologies such as SNOMED CT for clinical terms, LOINC for laboratory observations, MedDRA for adverse events, and/or the WHO Drug Dictionary for drug identification and classification. Using these standardized terminologies may enhance data consistency and facilitate interoperability with other systems. In some cases, the system may implement automatic data transformation rules to convert incoming data into the standardized format. For instance, if a module receives dates in various formats (e.g., MM/DD/YYYY, DD-MM-YYYY), the system may automatically convert these to a standard ISO 8601 format (YYYY-MM-DD) before storing in the centralised database.

In an embodiment, the data received via the one or more modules is saved in their corresponding table by using the patient identifier. The patient identifier may serve as a primary key in each data table, linking all data related to a specific patient across different modules. This identifier may be a unique, system-generated code to ensure patient privacy and comply with data protection regulations. These unique identifiers may be generated by the system for each entity involved in the clinical trial, such as studies, sites, subjects, and visits. The unique identifiers may serve as a common attribute that can be used to establish relationships between data entries in different modules. For instance, when a new patient is enrolled in the clinical trial, the system may generate a unique identifier for the patient. This unique identifier may then be associated with all data entries related to the subject across different modules. For example, the patient's demographic data entered in the EDC module, the patients'reported outcomes entered in the ePRO module, and the patients'randomization data entered in the IRT module may all be linked via the patient's unique identifier. In some cases, the system may store metadata alongside the clinical data, in the respective data table. This metadata may include information about the data source, timestamp of data entry, the user who entered the data, and any subsequent modifications. This additional information may enhance data traceability and support audit processes.

In an embodiment, the system may identify a type of clinical data and may activate a corresponding module to receive the clinical data. Upon receiving the data, the system may store the clinical data in the corresponding data table of that module, in the centralised database. For example, the system may first examine the file format and structure. The system detects that the file is a CSV (Comma-Separated Values) file with multiple columns and rows. The system then analyses the column headers and received data content. The system identifies fields such as ‘Patient ID’, ‘Visit Date’, ‘Systolic BP’, ‘Diastolic BP’, ‘Heart Rate’, and ‘Weight”’. Based on this analysis, the system recognizes that this data corresponds to vital signs collected during patient visits. The system may determine that this type of data should be handled by the Electronic Data Capture (EDC) module. The system activates the EDC module and passes the data file to the EDC module for processing. The EDC module then imports the data, mapping the fields to the appropriate sections of its Vital Signs electronic Case Report Form (eCRF). After processing, the system may be configured to store the data in the corresponding data table within the single database.

In an embodiment, at step 306, the system is configured to dynamically create at least one relational link between at least one module and one or more modules of the plurality of modules of the processing unit based on a set of preconfigured validation rules. The relational link is created by associating the corresponding data table of the at least one module and the one or more modules using the unique identifier. For example, when an adverse event is reported in the EDC module, the system may dynamically create a link via a corresponding entry in the medical coding module, using the unique identifier. In an embodiment, the one or more links are created based on preconfigured validation rules. These validation rules may define the relationships between different types of data entries across modules, specifying how data should be linked and synchronized based on certain criteria or conditions. The validation rules may include one or more of: definitions of what modules to be linked upon receiving a certain type of data, dependencies of modules on each other like which module(s) will be activated upon receiving a certain type of data, order in which the modules may be activated, order of synchronizing data in the data tables corresponding to the linked one or more modules and the like. These rules may help ensure data integrity, maintain protocol compliance, and streamline the clinical trial process. Here are some examples of preconfigured validation rules: 1. Module linking based on data type: When patient demographic data is entered in the EDC module, the system may automatically link to the ePRO, eConsent, and IRT modules. When an adverse event is reported in the EDC module, the system may create a link to the Coder module for proper coding. For example, when an adverse event term is entered in the EDC (e.g., ‘feeling dizzy’), the system automatically triggers a query to the Coder module. The Coder module, utilizing standardized dictionaries like MedDRA (Medical Dictionary for Regulatory Activities), WHO Drug, and the like, attempts to match the entered term with standardized medical terminology. If a match is found, the system creates a link between the original EDC entry and the standardized term in the Coder module. If multiple potential matches are found, the system may flag this for manual review by a qualified coder, while still maintaining the link between modules. 2. Module dependencies and activation: upon creation of a new patient record in the EDC module, the system may activate the eConsent module to initiate the informed consent process. After randomization data is entered in the IRT module, the system may activate the EDC module to allow entry of treatment-specific data. 3. Order of module activation: require eConsent module completion before allowing data entry in the EDC or ePRO modules. Mandate EDC module entry of eligibility criteria before activating the IRT module for randomization. 4. Data synchronization order: when updating patient weight in the EDC module, the system may first synchronize with the IRT module for dose calculations, then update the CTMS module for overall patient status. Upon entering a new adverse event in the EDC module, first synchronize with the Coder module for proper coding, then update the CTMS module for safety monitoring. 5. Conditional module linking: If a patient reports severe symptoms in the ePRO module, automatically link to the EDC module to trigger an unscheduled visit. When a protocol amendment is uploaded in the eTMF module, the system may create links to the EDC and eConsent modules to ensure updates to case report forms and informed consent documents. 6. Data-driven module activation: If lab results entered in the EDC module show values outside predefined ranges, the system may activate the adverse event form within the EDC module. When a patient completes a certain number of visits as recorded in the CTMS module, activate additional questionnaires in the ePRO module. 7. Synchronization based on data criticality: Prioritize real-time synchronization of safety data (e.g., adverse events) across EDC, Coder, and CTMS modules. Schedule non-critical data (e.g., quality of life questionnaires) for batch synchronization during off-peak hours.

Here are some more examples for the preconfigured validation rules. In some cases, the system may employ preconfigured validation rules that include definitions to identify the type of data being received and determine subsequent actions based on that data. These rules may play a crucial role in directing the flow of information and triggering appropriate processes within the clinical trial management system. Here's an elaboration on how these rules might function: 1. Data type identification: the system may analyze incoming data to determine its nature and relevance. For example: If the data includes specific biomarkers or diagnostic codes associated with cancer (e.g., elevated PSA levels, presence of BRCA mutations), the system may categorize the patient as a potential cancer trial participant. If the data indicates a diagnosis of type 2 diabetes along with HbA1c levels above 7%, the system may flag the patient for a diabetes-related trial. 2. Trial eligibility determination: based on the identified data type, the system may automatically assess the patient's eligibility for specific trials: For a breast cancer trial, if the data shows a female patient aged 40-65 with a newly diagnosed HER2-positive tumour, the system may flag the patient as potentially eligible. For a cardiovascular trial, if the data indicates a history of myocardial infarction and current use of specific medications, the system may mark the patient for further screening. 3. Randomization decisions: The validation rules may determine whether a patient should be sent for randomization based on specific criteria: In a cancer trial, if the patient's data shows completion of initial eligibility screening, signed informed consent (verified in the eConsent module), and baseline tumour measurements (recorded in the EDC module), the system may trigger the randomization process in the IRT module. For a diabetes trial, the system may initiate randomization only after confirming that the patient's HbA1c levels fall within the required range, there are no contraindicated medications (checked via the Coder module), and the patient has completed baseline quality of life questionnaires (verified in the ePRO module). 4. Module activation and data routing: The rules may dictate which modules should be activated or updated based on the received data: If the data indicates the occurrence of an adverse event, the system may activate the relevant forms in the EDC module, create an entry in the Coder module for proper coding, and update the CTMS module for safety monitoring. When data shows that a patient has completed a specific number of treatment cycles, the system may activate additional quality of life questionnaires in the ePRO module and schedule follow-up visits in the CTMS module. 5. Data-driven protocol adjustments: The validation rules may also facilitate protocol adjustments based on received data: In an adaptive trial design, if interim data analysis shows a particular treatment arm is underperforming, the system may adjust the randomization ratios in the IRT module to favour more effective treatment arms. If safety data indicates an increased frequency of a specific adverse event, the system may trigger updates to the informed consent documents in the eConsent module and modify the visit schedule in the CTMS module to include additional safety assessments. By implementing these types of preconfigured validation rules, the system can automate many aspects of trial management, ensure protocol adherence, and facilitate rapid, data-driven decision-making. This approach may help to streamline trial operations, reduce manual errors, and ensure that patients are appropriately managed throughout the clinical trial process.

In an embodiment, the system is configured to continuously monitor adherence to the preconfigured validation rules across the one or more modules. For example, the system implements a background process that constantly checks the state and interactions of all linked modules. As data moves between modules, the system tracks these transfers and compares them against the preconfigured validation rules. The system monitors the timing of data transfers and actions to ensure they occur in the correct sequence. The system verifies that data remains consistent across linked modules.

In an embodiment, at step 308, the system is configured to synchronize, in real time, information across the linked one or more modules based on the received clinical data from the at least one module. The synchronizing comprises automatically populating the data tables of the linked one or more modules based on the clinical data received by the at least one module and the set of preconfigured validation rules. For example, the synchronization process may involve several steps: when clinical data is received by a module, it triggers the synchronization process. The system evaluates the received data against a set of pre-configured validation rules to determine which other modules need to be activated and linked. The system then maps the received data to the appropriate fields in the linked module. The system automatically populates the data tables of the linked modules with the relevant information, in the centralised database. The system performs consistency checks to ensure the synchronized data is accurate across all modules. Here's an example of how this process works: consider a clinical trial for a new diabetes medication where the Electronic Data Capture (EDC) module, Interactive Response Technology (IRT) module, and Electronic Patient-Reported Outcome (ePRO) module are linked. A study coordinator enters new data into the EDC module during a patient's visit. The data includes: Patient ID: 12345, Visit Date: 2023-06-1, Fasting Blood Glucose: 180 mg/dL, HbA1c: 8.2%, Weight: 82 kg, upon receiving this data, the system evaluates it against the preconfigured validation rules. Lets'say one rule state that if a patient's HbA1c is above 8%, the IRT module should be notified for potential dose adjustment, and the ePRO module should schedule additional glucose monitoring. Based on this rule, the system initiates the synchronization process: For the IRT module: the system maps the relevant data (Patient ID, Visit Date, HbA1c) to the IRT module's data structure. The system automatically populates the IRT module's data table with this information. The IRT module then flags this patient identifier for potential dose adjustment review. b) For the ePRO module: the system maps the relevant data (Patient ID, Visit Date, HbA1c) to the ePRO module's data structure. The system automatically populates the ePRO module's data table with this information, in the centralised module. The ePRO module then updates the patient's questionnaire schedule to include daily glucose monitoring for the next week. The system performs consistency checks to ensure the synchronized data matches across all modules. For example, the system verifies that the Patient ID and Visit Date are identical in the EDC, IRT, and ePRO modules for this particular data entry. If any inconsistencies are detected during these checks, the system may flag them for review by the team. This real-time synchronization ensures that all modules have the most up-to-date information, enabling timely decision-making and maintaining data consistency across the entire clinical trial management system. The use of preconfigured validation rules allows for automated, intelligent updates across modules based on the specific requirements of the clinical trial protocol.

In an embodiment, at step 310, the system is configured to generate one or more metrics corresponding to the patient identifier based on the synchronized information, from the data tables of the single database, corresponding to linked one or more modules. For example, the system may generate metrics in several categories: 1. Patient-specific metrics: adherence to treatment regimen, frequency and severity of adverse events, changes in key clinical parameters over time, quality of life scores, visit attendance rate. 2. Study-wide metrics: enrolment rate, dropout rate, overall adverse event frequency, protocol deviation frequency, data query rate and resolution time. 3. Site-specific metrics: patient recruitment rate, data entry timeliness, query response time, protocol adherence rate. 4. Efficacy metrics: primary endpoint achievement rate, secondary endpoint trends, subgroup analysis results. 5 safety metrics: serious adverse event frequency, adverse event patterns, laboratory abnormality trends. 6. Data quality metrics: missing data percentage, data entry error rate, source data verification findings. 7. Operational metrics: time to site activation, time to first patient enrolled, time to database lock, and the like. The system may calculate these metrics using various statistical methods, including descriptive statistics, trend analysis, and more complex statistical models. In some cases, the system may employ machine learning algorithms to identify patterns or predict outcomes based on the synchronized data. Here's an example to understand this process: For example, consider a patient named Sarah Johnson, with the unique identifier PTN-5678, enrolled in a phase II clinical trial for a new hypertension medication. The system may generate the following metrics based on synchronized information from multiple linked modules: 1. From the EDC module: Blood pressure readings over time, Medication dosage adjustments. 2. From the ePRO module: Quality of life scores, Reported side effects. 3. From the IRT module: Randomization group, Drug dispensation history. 4. From the Coder module: Coded adverse events, Concomitant medications. 5. From the CTMS module: Visit compliance, Protocol deviations. Based on this synchronized information, the system may generate the following metrics for Sarah: 1. Treatment Efficacy: Average systolic blood pressure reduction: 15 mmH, Percentage of blood pressure readings within target range: 75% 2. Safety Profile: Number of reported adverse events: 2 (mild headache, transient dizziness), Severity distribution of adverse events: 100% mild 3. Patient Compliance: Medication adherence rate: 98% (based on ePRO reports and drug dispensation records), Visit attendance rate: 100% 4. Quality of Life Impact: Change in quality of life score from baseline: +15%, Improvement in reported energy levels: 30%. 5. Protocol Adherence: Number of protocol deviations: 0, Completion rate of required assessments: 100%. 6. Trial Progress: Current trial phase: Week 12 of 24, Next scheduled visit: 3 days from now. By generating these metrics, the system provides a comprehensive overview of Sarah's progress in the trial, combining data from multiple modules to create a holistic view of her treatment response, safety profile, and overall trial experience. This information may help the system to make informed decisions about Sarah's continued participation in the trial and contribute to the overall analysis of the investigational medication's efficacy and safety.

The system may present these metrics through interactive dashboards, allowing users to drill down into specific data points, compare across patient subgroups, or view trends over time. In some cases, the system may also generate alerts based on predefined thresholds, such as notifying the doctors, site-coordinators and the like if the serious adverse event rate exceeds a certain percentage. These comprehensive metrics, generated from synchronized data across all linked modules, may provide trial managers with a holistic view of both individual patient progress and overall trial performance. This may facilitate data-driven decision-making, early identification of potential issues, and ultimately contribute to more efficient and effective clinical trial management.

In an embodiment, at step 312, the system may be configured to displaying, on a user interface communicably coupled to the processing unit, at least one of the generated one or more metrics corresponding to the patient identifier. This feature may provide clinical trial staff, investigators, and managers with easy access to critical patient-specific information, facilitating informed decision-making and efficient trial management. The user interface may present the metrics in various formats, including numerical displays for precise quantitative metrics, charts and graphs for visualizing trends over time, color-coded indicators for quick status assessments, and interactive dashboards for exploring data in depth. The system may allow users to customize the display based on their role and preferences, with different views tailored for clinical research coordinators, principal investigators, data managers, and project managers. The user interface may also incorporate features such as drill-down capabilities, comparison tools, trend analysis, and alert indicators to highlight metrics that fall outside predefined ranges. For example, in a clinical trial for a new rheumatoid arthritis treatment, the user interface might display a comprehensive patient overview including efficacy metrics like DAS28 scores and joint counts, safety metrics such as adverse events and lab values, treatment adherence data, quality of life scores, and data quality indicators. By presenting these metrics in an intuitive and interactive format, the system may enable trial staff to quickly assess a patient's status, identify areas of concern, and take appropriate actions, ultimately contributing to improved patient care, better protocol adherence, and more efficient trial management.

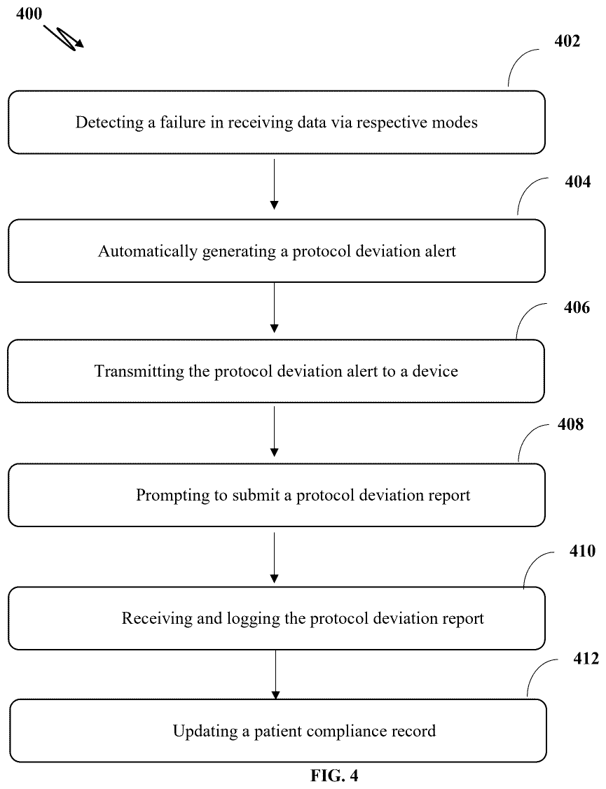

In an embodiment, the system may include a protocol adherence mechanism. This mechanism is designed to detect, report, and manage instances where a patient fails to comply with the prescribed protocol of the clinical trial. The system continuously monitors patient activity within the system. Referring to FIG. 4 now, the system at step 402 is configured to detect when a patient fails to enter data via any of the modes disclosed above in the disclosure. Let's understand with the help of an example, where the data is entered in a module via for example, a required form associated with any module in the system, such as the Electronic Data Capture (EDC) module or the eConsent module to receive the patient data. This detection is performed in real-time, allowing for immediate action. Upon detecting a failure to complete a required form, the system at step 404 automatically generates a protocol deviation alert. This alert is a digital notification containing relevant information about the deviation, including but not limited to patient identifier, module identifier, form identifier, timestamp of the detected deviation, nature of the deviation (e.g., ‘failure to complete required form’). Hence, this mechanism may involve time-based form activation, where forms for capturing trial data are activated based on the timing specified in the trial protocol. For instance, if a trial protocol specifies that a certain form should be completed seven days after a patient's initial visit, the system may automatically enable this form seven days after the initial visit is recorded in the system. This time-based form activation may help ensure that trial activities are conducted in accordance with the trial protocol, potentially improving trial compliance and data quality. In an embodiment, the system's protocol adherence mechanism may ensure that trial activities are conducted in accordance with the trial protocol. This may involve time-based form activation, where forms for capturing trial data are activated based on the timing specified in the trial protocol. By ensuring protocol adherence, the platform may reduce the need for manual protocol checks and minimize the risk of protocol deviations, potentially improving trial compliance and data quality.

In an embodiment, the system transmits the generated protocol deviation alert to a patient interface device at step 406. The device may be a smartphone, tablet, computer, or any other suitable electronic device capable of receiving and displaying notifications. The transmission is performed securely, ensuring the confidentiality of patient information. In an embodiment, upon receiving the alert, the patient interface device prompts the patient to submit a protocol deviation report at step 408. This prompt is displayed on the device's screen and may include: a brief explanation of the detected deviation, instructions for submitting a deviation report, a direct link or button to access the secure electronic submission system. In an embodiment, the system may incorporate a secure electronic submission system for protocol deviation reports. This system may provide a user-friendly interface for patients to input deviation details and ensures data encryption during transmission. The system may authenticate the patient's identity before allowing submission and guides the patient through a structured report format to ensure all necessary information is captured.

In an embodiment, when a patient submits a protocol deviation report through the secure electronic submission system, the system receives the report in real-time at step 410. The system logs the report in a dedicated database within the platform. The system may associate the report with the patient's unique identifier and the specific clinical trial. The system may timestamp the receipt of the report for audit trial purposes. In an embodiment, upon successful receipt and logging of the protocol deviation report, the system at step 412 automatically updates the patient's compliance record. This update includes recording the occurrence of the protocol deviation, noting the timely submission of the deviation report, calculating and updating compliance metrics based on the nature and frequency of deviations, and flagging the patient record for review by the clinical trial staff if necessary.

The protocol adherence mechanism within the system ensures timely detection and reporting of protocol deviations, facilitates patient communication regarding these deviations, and maintains accurate, up-to-date patient compliance records. This mechanism enhances the overall integrity of the clinical trial data and supports regulatory compliance by providing a comprehensive audit trail of protocol deviations and their management. For example, the protocol adherence mechanism may integrate with wearable devices for real-time monitoring of adherence. For instance, if a trial protocol requires patients to take a certain medication at specific times, the system may integrate with a wearable device that tracks medication intake. If the wearable device detects that a patient has not taken the medication at the specified time, the system may generate a notification to remind the patient or alert the trial staff. This real-time monitoring of adherence may help ensure that patients follow the trial protocol, potentially improving trial compliance and data quality.

The implementation of the data transmission and storage processes across modules offers several key advantages: 1. Data Consistency: By automatically sharing and storing critical data across modules, the system ensures consistency of information throughout the platform. 2. Reduced Data Entry: The need for manual re-entry of data in different modules is eliminated, reducing the potential for human error and improving efficiency. 3. Real-time Data Availability: As data is transmitted and stored across modules in real-time, all parts of the system have immediate access to the most current information. 4. Enhanced Data Integrity: The automated nature of the data transmission and storage processes reduces the risk of data corruption or loss during manual transfers. 5. Improved Compliance: By ensuring that consent data is readily available across all relevant modules, the system supports better adherence to regulatory requirements regarding informed consent in clinical trials. 6. Streamlined Workflows: With critical data available across modules, the system can more effectively automate and manage workflows that span multiple aspects of the clinical trial process.

In an embodiment, each of the module corresponds to a distinct phase of the clinical trial. In an embodiment, the one or more modules are interconnected within the processing unit. For example, the interconnection of modules within the system is a key feature that enables seamless data flow and process continuity. In an embodiment the one or more modules may have a shared data access. The one or more modules may have access to the centralized database, allowing real-time data sharing. The changes in one or more modules are immediately reflected across the system. In an embodiment, the one or more modules may have workflow integration. The one or modules are linked through predefined workflows. The completion of tasks in one of the modules may trigger actions in subsequent modules. In an embodiment, the one or more modules may be enabled by single sign-on functionality which allows seamless navigation between modules.

In an embodiment, the system may monitor adherence to the predefined clinical trial protocols across the one or more modules. The system may continuously track all activities and data entries across the one or more modules, comparing them against the predefined protocol parameters. This real-time monitoring system utilizes a combination of automated checks and user-defined rules to identify any deviations from the protocol. For instance, if a patient visit occurs outside the predefined protocols, the system may flag this as a potential protocol violation. In an embodiment, the system may automatically check that all required data points, as specified in the protocol, are collected within each module. The system also ensures that data entries fall within predefined ranges or match expected formats, triggering alerts for any out-of-range or inconsistent data. In an embodiment, the system has a hierarchical alert system that notifies relevant personnel of potential protocol deviations based on their severity. Minor discrepancies may generate warnings within the system, while significant violations may trigger immediate notifications to study coordinators or principal investigators. This tiered approach allows for prompt corrective actions and helps maintain the integrity of the trial. By integrating this monitoring capability across all functional modules, the system ensures consistent protocol adherence throughout the entire clinical trial process, from patient enrolment through data collection and analysis. This comprehensive approach significantly enhances the quality and reliability of the trial data, ultimately supporting the validity of the study results.

In an embodiment, the system is configured to identify deviations based on the monitoring. The system may dynamically adjust access permissions to at least one of the modules based on the identified deviations. When a deviation is detected, the system may log and categorized them based on its nature and severity. This categorization may include minor protocol deviations, significant protocol violations, or critical safety issues. For example, upon identifying a deviation, the system initiates a dynamic adjustment of access permissions: For severe deviations, the system may immediately restrict access to certain modules or functions to prevent further protocol violations. For example, if a patient is found to be ineligible after initial screening, access to the randomization module for that patient may be automatically revoked. In an embodiment, the system may increase the level of authorization required for certain actions. For instance, data entry in a particular module might require additional approvals or review steps following a deviation. In an embodiment, in cases of significant deviations, entire modules might be temporarily locked for the affected patient or site until the issue is resolved and documented. In an embodiment, the system may initiate mandatory review processes, granting temporary elevated access to quality control personnel or study monitors to investigate and resolve the deviation. In an embodiment, for minor deviations, the system might impose time-limited restrictions that automatically expire after a set period, assuming no further issues are detected.

In an exemplary embodiment, the method as disclosed in FIGS. 3A and 3B may work as follows: Consider a phase III clinical trial for a new type 2 diabetes medication. The trial involves multiple sites and uses several modules within a unified clinical trial management system. Here's how the method might work in practice: 1. A patient, John Doe, is enrolled in the trial and assigned a unique patient identifier: PTN-1234. 2. During a routine visit, a nurse enters or the patient enters, John's clinical data into the Electronic Data Capture (EDC) module: Blood glucose: 165 mg/dL, HbA1c: 7.8%, Weight: 88 kg, Blood pressure: 138/85 mmHg 3. The system stores this data in the EDC module's data table within the single database, associating it with the patient identifier PTN-1234. 4. Based on preconfigured validation rules, the system creates relational links between the EDC module and other relevant modules: Interactive Response Technology (IRT) module for medication dispensing and randomization. Electronic Patient-Reported Outcome (ePRO) module for patient questionnaires. eConsent module for managing informed consent. Coder module for adverse event and medication coding. Clinical Trial Management System (CTMS) module for overall trial management. 5. The system synchronizes the information across these linked modules: The IRT module is updated with John's current weight to calculate the correct medication dose. The ePRO module is notified to schedule a quality of life questionnaire based on the HbA1c result. The eConsent module is checked to ensure John's consent is still valid for the current phase of the trial. The Coder module is updated with any new medications or adverse events reported during the visit. The CTMS module is updated with John's visit completion status and any protocol deviations. 6. The system generates metrics based on the synchronized information: Treatment adherence: 95% (based on ePRO data), HbA1c change from baseline:-0.7%, Weight change from baseline: −2.3 kg, Blood pressure control: Improved (systolic decreased by 7 mmHg), Adverse event frequency: 1 mild event reported (from Coder module), Visit compliance: 100% (from CTMS module) 7. These metrics are displayed on a user interface accessible to the study coordinator: A dashboard shows John's key metrics compared to study averages. A graph displays John's HbA1c trend over time. An alert indicates that John's blood glucose is above the target range. A summary of John's reported adverse events and concomitant medications is shown. The status of John's informed consent and upcoming visit schedule is displayed. This example demonstrates how the method securely manages clinical data, ensures real-time synchronization across modules, and provides valuable insights to trial staff, all while maintaining data integrity and patient confidentiality across multiple aspects of the clinical trial process.

Thus, the system is designed to maintain data integrity across modules without manual data transfer between these modules. This is achieved through the use of the centralized/single database and the mapping component, which together ensure that all modules are working with the same, up-to-date set of trial data. This feature may help to reduce the risk of data loss or inconsistencies, which can compromise the validity of clinical trial results. The system leverages this architecture to maintain referential integrity across all modules. Any updates or modifications to data in the one module are automatically reflected in all linked modules through these relational connections. This ensures that all parts of the system are working with the most current and accurate information at all times. Furthermore, this method of data management allows for complex, multi-directional relationships between modules. For example, adverse event data entered in the EDC can trigger the creation of a safety report in a separate module, while simultaneously updating the patient's status in the IRT module. All of these actions are accomplished through the manipulation of relational links rather than by duplicating or physically transferring data. This approach not only optimizes data accessibility and consistency but also enhances system performance by reducing data redundancy and minimizing the need for resource-intensive data transfers between modules. It provides a robust foundation for implementing sophisticated business rules and workflows that span multiple aspects of the clinical trial process, all while maintaining the highest standards of data integrity and security.

Exemplary embodiments discussed above may provide certain advantages. Though not required to practice aspects of the disclosure, these advantages may include those provided by the following features. The system effectively addresses the challenges associated with data transfer between disparate systems by implementing the following solutions:
1. Centralized Data Management: The system consolidates all trial-related data into a single, centralized system. By centralizing data management, it eliminates the need for transferring data between separate systems, which reduces the risk of data loss and corruption. This central repository ensures that all data is consistently stored, maintained, and updated in one location, thereby enhancing data integrity and security. 2. Automated Data Synchronization: The system can utilize automated data synchronization processes to ensure that all data is accurately and efficiently updated in real-time. Automated synchronization reduces the need for manual data entry and transfers, minimizing the risk of human error and inconsistencies. It ensures that any changes or updates in the data are immediately reflected across the entire system, maintaining accurate and up-to-date information. 3. Integrated Security Measures: The system incorporates robust security measures to protect patient data and maintain compliance with regulatory standards. Advanced encryption, access controls, and authentication protocols are employed to safeguard sensitive information from unauthorized access and breaches. By integrating these security features directly into the platform, it ensures that patient data remains confidential and secure throughout its lifecycle. 4. Real-Time Data Validation and Error Checking: The system includes real-time data validation and error-checking functionalities. These tools automatically detect and correct data entry errors, inconsistencies, or anomalies before data is finalized. By ensuring data accuracy and completeness, the platform helps prevent the transmission of incomplete or corrupted data, which is crucial for making informed and accurate treatment decisions. 5. Comprehensive Auditing and Monitoring: The system provides comprehensive auditing and monitoring capabilities to track data access, modifications, and transfers. Detailed logs and reports are generated to provide transparency and accountability. This functionality helps identify and address any issues or discrepancies promptly, ensuring that data remains reliable and that any potential problems are addressed swiftly. 6. Streamlined Data Integration: By integrating various trial functions-such as EDC, CTMS, eTMF, IRT, eConsent, and ePRO-into a single platform, the need for cross-system data transfers is eliminated. This integrated approach simplifies data management and ensures that all relevant information is seamlessly interconnected. It also facilitates easier and more accurate data analysis, reducing the risk of miscommunication or data misalignment. 7. Enhanced Workflow Automation: The system automates workflows and processes, such as data collection, consent management, and randomization. This automation ensures that all procedures are consistently followed and that data is collected and processed efficiently. By reducing manual interventions and streamlining operations, the platform enhances overall trial efficiency and accuracy. In summary, the system addresses the problems of data transfer, loss, and fragmentation by centralizing data management, automating synchronization, integrating security measures, validating data in real-time, providing comprehensive auditing, and streamlining workflows. This integrated approach enhances data accuracy, security, and efficiency, ultimately leading to more reliable and effective clinical trial management.

Although implementations for methods and systems for managing a clinical trial of patients have been described in language specific to structural features and/or methods, it is to be understood that the appended claims are not necessarily limited to the specific features or methods described. Rather, the specific features and methods are disclosed as examples of implementations for classifying components of a product.

1. A method for securely managing clinical data associated with a patient, corresponding to a patient identifier, enrolled in a clinical trial, the method comprising:
receiving, via a respective mode activated for a predefined time window, the clinical data via at least one module out of a plurality of modules of a processing unit, wherein each of the plurality of modules is associated with a corresponding data table within a centralized database communicably coupled to the processing unit;
detecting, in real-time, a failure in the received clinical data via the respective mode associated with the at least one module,
wherein upon detection of the failure:
automatically generating, in response to the detected failure, a protocol deviation alert;
transmitting the protocol deviation alert to a user device;
receiving a protocol deviation report from the user device, wherein the protocol deviation alert is received in an encrypted format; and
updating the clinical data related to the patient identifier based on the protocol deviation report;
storing the clinical data in a format against the patient identifier in the corresponding data table of the at least one module in the centralized database, wherein the format is unique to the data table of the at least one module;
dynamically creating at least one relational link between the at least one module and one or more modules of the plurality of modules of the processing unit based on a set of preconfigured validation rules, wherein creating the relational link comprises associating the corresponding data tables of the at least one module and the one or more modules using the unique identifier, wherein the data tables corresponding to the linked one or more modules are synchronized automatically in real-time, based on the preconfigured validation rules, on receiving an update in the clinical data in any one of the linked one or more modules, and wherein the set of preconfigured validation rules define an order of synchronizing data in the data tables corresponding to the linked one or more modules;
wherein before synchronizing the data in the corresponding data tables of the linked one or more modules:
activating, based on the set of preconfigured validation rules, at least the one more modules of a plurality of modules of the processing unit to receive the data; and
converting, the data in a format corresponding to each data table of the linked one or more modules;
synchronizing, in real time, the data across the linked one or more modules, wherein synchronizing comprises automatically populating the data tables of the linked one or more modules based on the clinical data received by the at least one module and the set of preconfigured validation rules;
generating one or more metrics corresponding to the patient identifier based on the synchronized information, from the data tables of the centralized database, corresponding to linked one or more modules; and
displaying, on a user interface communicably coupled to the processing unit, at least one of the generated one or more metrics corresponding to the patient identifier.

2. (canceled)

3. (canceled)

4. The method of claim 1, wherein each module of the plurality of the modules corresponds to a specific clinical trial function.

5. The method of claim 1, wherein the set of preconfigured validation rules defines inter-dependencies amongst the plurality of modules of the processing unit.

6. The method of claim 1, wherein the set of preconfigured validation rules define an order of synchronizing data in the data tables corresponding to the linked one or more modules.

7. The method of claim 1 further comprises:
prompting, via a device, to submit a protocol deviation report;
receiving and logging the protocol deviation report in the centralized database; and
updating a patient compliance record based on the received protocol deviation report.

8. The method of claim 7 further comprises:
wherein the time window is activated for a predetermined time period.

9. A system for securely managing clinical data associated with a patient enrolled in a clinical trial, the system comprising:
a memory storing one or more instructions;
a processor communicatively coupled with the memory, wherein upon execution of the stored one or more instructions the processor is configured to:
receive, via a respective mode activated for a predefined time window, the clinical data via at least one module out of a plurality of modules of a processing unit, wherein each of the plurality of modules is associated with a corresponding data table within a centralized database communicably coupled to the processing unit;
detect, in real-time, a failure in the received clinical data via the respective mode associated with the at least one module, wherein upon detection of the failure:
automatically generate, in response to the detected failure, a protocol deviation alert;
transmit the protocol deviation alert to a user device;
receive a protocol deviation report from the user device, wherein the protocol deviation alert is received in an encrypted format; and
update the received clinical data related to the patient identifier based on the protocol deviation report;
store the clinical data in a format against the patient identifier in the corresponding data table of the at least one module in the centralized database, wherein the format is unique to the data table of the at least one module;
dynamically create at least one relational link between the at least one module and one or more modules of the plurality of modules of the processing unit based on a set of preconfigured validation rules, wherein creating the relational link comprises associating the corresponding data tables of the at least one module and the one or more modules using the unique identifier, wherein the data tables corresponding to the linked one or more modules are synchronized automatically in real-time, based on the preconfigured validation rules, on receiving an update in the clinical data in any one of the linked one or more modules, and wherein the set of preconfigured validation rules define an order of synchronizing data in the data tables corresponding to the linked one or more modules;
wherein before synchronizing the data in the corresponding data tables of the linked one or more modules:
activate, based on the set of preconfigured validation rules, at least the one more modules of a plurality of modules of the processing unit to receive the data; and
convert, the data in a format corresponding to each data table of the linked one or more modules;
synchronize, in real time, the data across the linked one or more modules wherein synchronizing comprises automatically populating the data tables of the linked one or more modules based on the clinical data received by the at least one module and the set of preconfigured validation rules and;
generate one or more metrics corresponding to the patient identifier based on the synchronized information, from the data tables of the centralized database, corresponding to linked one or more modules; and
display, on a user interface communicably coupled to the processing unit, at least one of the generated one or more metrics corresponding to the patient identifier.

10. (canceled)

11. (canceled)

12. The system of claim 9, wherein each module of the plurality of the modules corresponds to a specific clinical trial function.

13. The system of claim 9, wherein the set of preconfigured validation rules defines inter-dependencies amongst the plurality of modules of the processing unit.

14. The system of claim 9, wherein the set of preconfigured validation rules define an order of synchronizing data in the data tables corresponding to the linked one or more modules.

15. The system of claim 9, wherein the processor is further configured to:
prompt, via a device, to submit a protocol deviation report;
receive and log the protocol deviation report in the centralized database; and
update a patient compliance record based on the received protocol deviation report.

16. The system of claim 15, wherein the processor is further configured to:
wherein the time window is activated for a predetermined time period.
